(19) United States
(12) Patent Application Publication
(10) Pub. No.: US 2018/0245463 A1
(43) Pub. Date: Aug. 30, 2018

(54) METHOD FOR DETERMINING A WATER CUT OF AN OIL-WATER MIXTURE PRODUCED FROM AN OIL WELL

(71) Applicant: SCHLUMBERGR TECHNOLOGY CORPORATION, Sugar Land, TX (US)

(72) Inventors: Valery Vasilievich Shako, Moscow (RU); Vyacheslav Pavlovich Pimenov, Moscow (RU); Bertrand Theuveny, Moscow (RU); Maria Viktorovna Sidorova, Moscow (RU); Alexander Alexandrovich Burukhin, Moscow (RU); Nikita Ilyich Ryzhikov, Moscow (RU)

(21) Appl. No.: 15/655,937

(22) Filed: Jul. 21, 2017

(30) Foreign Application Priority Data
Jul. 21, 2016 (RU) 2016129942

(51) Int. Cl. E21B 49/08; E21B 47/06; G01N 9/26; G01N 33/28

(52) U.S. Cl. CPC E21B 49/08; E21B 47/06; E21B 2049/085; G01N 9/26; G01N 33/2847; E21B 47/065

(57) ABSTRACT

For determining a water cut of an oil-water mixture produced from an oil well at least one Venturi tube is placed in a well, through which an oil-water mixture, produced from a selected well segment, enters the borehole. During production process, pressure at a Venturi tube inlet and in a Venturi tube throat are measured, and flow temperature of the produced oil-water mixture at the Venturi tube inlet and the temperature of the Venturi tube wall in the Venturi tube throat are measured. Based on the results of the pressure and temperature measurements, the water cut of the oil-water mixture produced from the selected well segment is determined.

### CROSS-REFERENCE TO RELATED APPLICATION

This application claims priority to Russian Application No. 2016129942 filed Jul. 21, 2016, which is incorporated herein by reference in its entirety.

### BACKGROUND

The present disclosure relates to methods for determining composition of an oil-water mixture in a well, in particular, to methods using measurements of flow parameters of a produced fluid in a Venturi tube, through which the oil-water mixture, produced from a selected well segment, enters a borehole.

The measurement of the composition of a multi-phase flow in a borehole is an important problem in controlling and monitoring of production. It is necessary mainly in highly productive wells having complex completion, particularly, in multilateral wells and wells with inflow control devices enabling optimization of the oil production by reducing production rate or shut off production from the well segments having high water cut.

In downhole multi-phase flowmeters, a combination of a Venturi tube (pressure drop is measured by in a Venturi tube throat) and devices for measuring multiphase mixture properties is usually used. These devices may be a gamma-ray densitometer (e.g. U.S. Pat. No. 6,776,054), a capacitive phase composition meter (US 20120041681), and the like.

Thus, the application US 20120041681 describes the use of a capacitive meter of phase composition. The main drawback of this method lies in low accuracy of the water cut measurement at its high values (more than 30%).

### SUMMARY

The disclosure provides for increased accuracy and reliability of determining a water cut of the produced oil-water mixture through the whole range of values of this measure.

In accordance with the proposed method, at least one Venturi tube is placed in a well, through which an oil-water mixture, produced from a selected well segment, enters a borehole. During production process, pressure at a Venturi tube inlet and in a Venturi tube throat are measured, and flow temperature of the produced oil-water mixture at the Venturi tube inlet and the temperature of the Venturi tube wall in the Venturi tube throat are measured. Based on results of the pressure and temperature measurements, a water cut of the oil-water mixture produced from the selected well segment is determined.

In accordance with one embodiment of the disclosure, additionally pressure and temperature of the produced oil-water mixture are further measured downstream of the Venturi tube outlet. At the same time, temperature sensors arranged at a distance of 10-20 diameters of the Venturi tube downstream of the Venturi tube throat can be used to measure the temperature downstream of the Venturi tube outlet.

In accordance with yet another embodiment of the disclosure, all temperature measurements are performed during production rate change or production shut off.

According to one more embodiment of the disclosure, temperature sensors arranged at a distance of 1-2 diameters of the Venturi tube before the Venturi tube constriction are used to measure the temperature at the Venturi tube inlet.

### BRIEF DESCRIPTION OF DRAWINGS

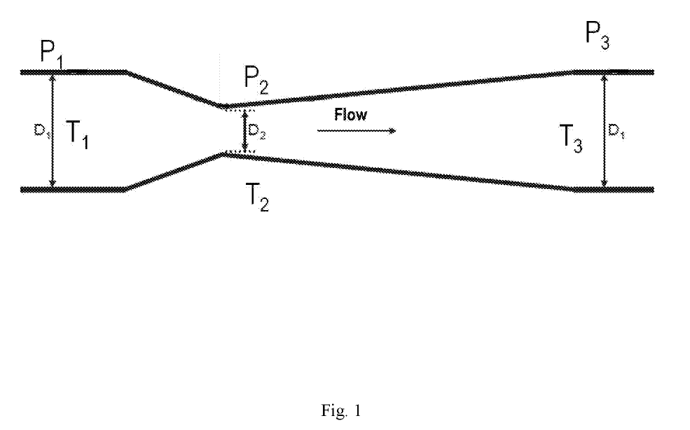

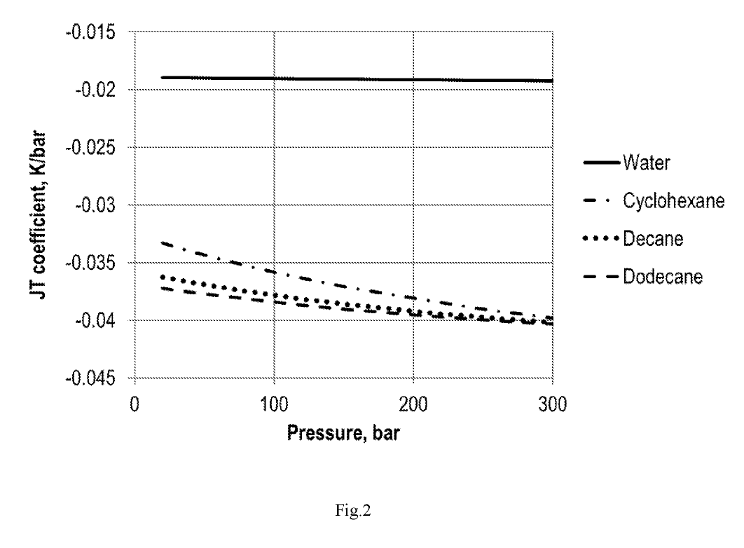

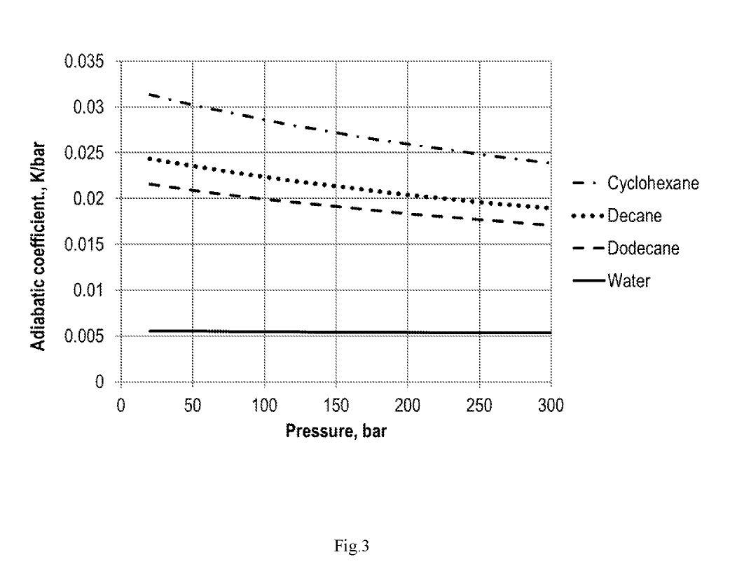

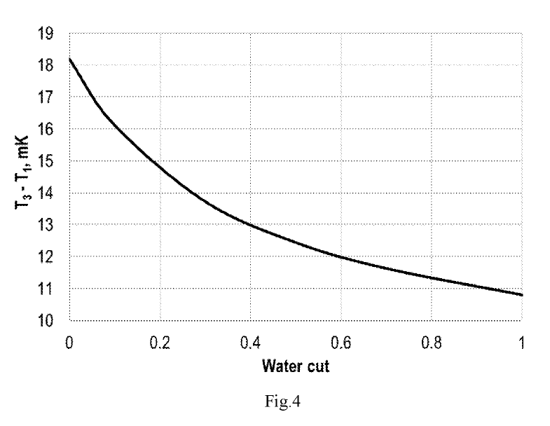

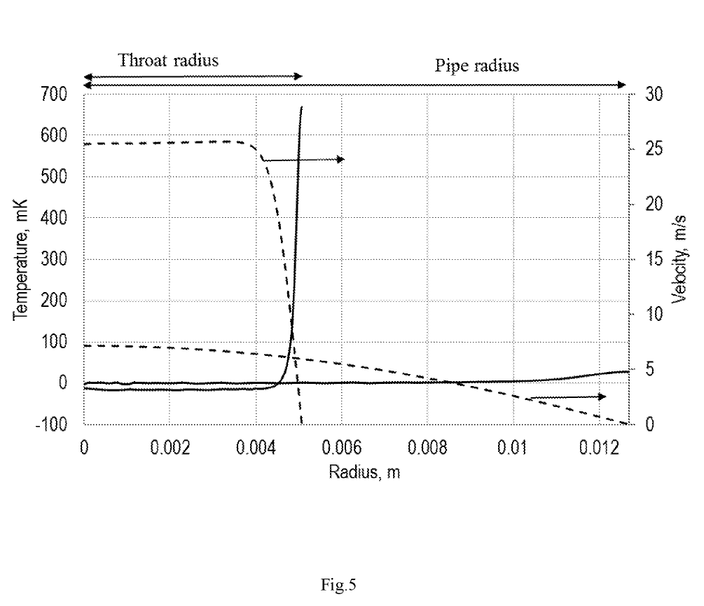

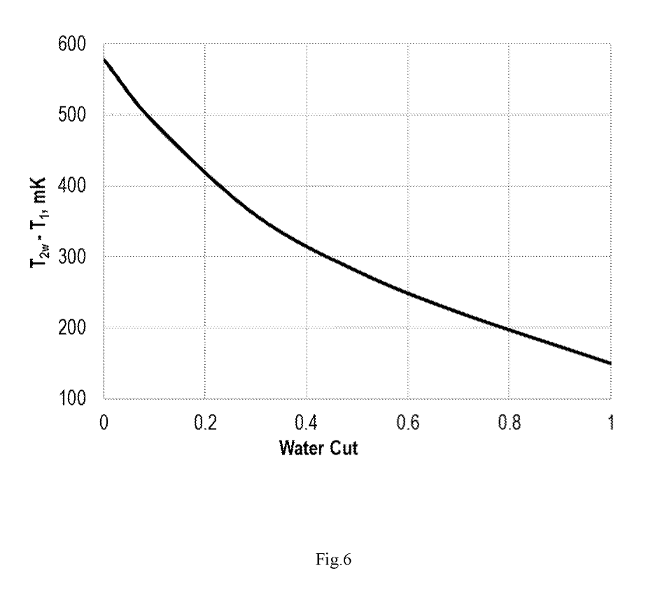

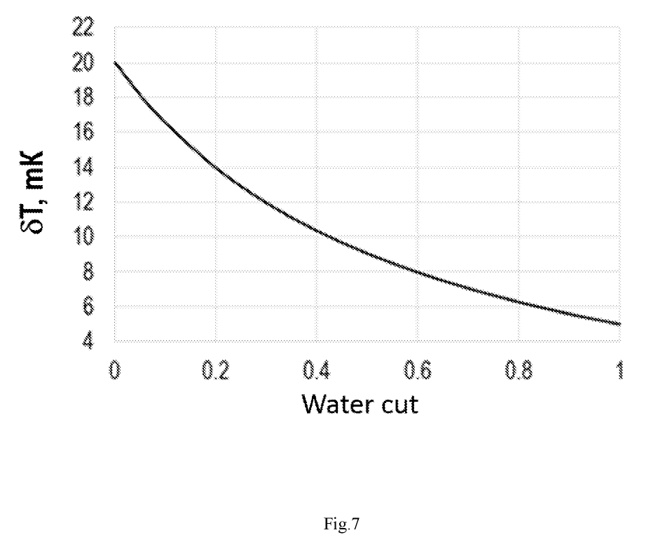

The disclosure is explained by the drawings, in which FIG. 1 shows a scheme of a Venturi tube, FIG. 2 shows relationship of Joule-Thompson coefficients for water and certain hydrocarbons versus pressure at a temperature of 80° C.; FIG. 3 shows relationship of adiabatic coefficients for water and certain hydrocarbons versus pressure at a temperature of 80° C.; FIG. 4 shows relationship of heating of the oil-water mixture downstream of the Venturi tube outlet due to the Joule-Thomson effect versus water cut, FIG. 5 shows the calculated radial distribution of velocities (dashed lines) and temperature at the beginning of constriction and in the Venturi tube throat, FIG. 6 shows relationship of the temperature rise of the wells in the Venturi tube throat versus water cut, FIG. 7 is the calculated relationship of the amplitude of adiabatic temperature changes versus water cut for the pressure change δP=10 bar.

### DETAILED DESCRIPTION

The method comprises pressure measurements as well as temperature measurements indicative of the phase composition of the produced mixture, in a Venturi tube. At least one Venturi tube, through which the oil-water mixture enters a borehole from a selected well segment, is placed in a well. The number of Venturi tubes is determined by the number of well segments for which it is necessary to determine the water cut of the produced oil-water mixture. The water cut of the produced oil-water mixture is determined by measuring pressure and temperature, which is important for long-term monitoring of oil production, as the modern pressure and temperature sensors can operate for more than 10 years in conditions existing in a borehole. The proposed method can be used in combination with the known methods, which makes it possible to increase accuracy of determining the water cut of the produced oil-water mixture through the whole range of values of this measure.

In accordance with the disclosure, in order to determine the water cut, the following temperature effects in the stream of the oil-water mixture produced from the selected segment are used, which depend on the oil-water mixture composition:
heating of the flow in the near-wall region and the walls in the Venturi tube throat due to viscous dissipation effects,
heating of the flow of the oil-water mixture due to irreversible pressure drop when the flow brakes after having been passed through the Venturi tube (Joule-Thompson effect),
changes in flow temperature caused by sudden pressure changes in the well, which depend on the composition of the water-oil mixture.

A scheme of a Venturi tube is shown in FIG. 1. Here (1) is a Venturi tube inlet, (2) is a throat, (3) is a Venturi tube outlet, T_{2w} is a wall temperature in the Venturi tube throat.

Pressure measurements are carried out by means of pressure sensors (for example, GE UNIK 5000 electronic absolute pressure sensors), and temperature measurements—by means of high-sensitive temperature sensors, for example, calibrated Hayashi Denko CRZ-1632-100-A-1 thin-film platinum resistance thermometers. The wall temperature in the Venturi tube throat can be measured through a channel drilled perpendicular to the axis of the tube in which the temperature sensor was located. For sealing and thermal contact, this channel was filled with a heat-conducting polymer.

As it follows from the one-dimensional equations of the amount of motion and energy, the flow temperature in the Venturi tube is determined by the following expression:

$$T ( x ) = {T}_{1} - η \cdot [ {P}_{1} - P ( x ) ] + \frac{1}{ρ {c}_{p}} \cdot Δ P ( x ) = {T}_{1} - η \cdot \frac{ρ}{2} \cdot [ {V ( x )}^{2} - {V}_{1}^{2} ] + ( - {μ}_{JT} ) \cdot Δ P ( x ) \qquad ( 1 )$$

where P₁, P₂ and P₃ are static pressure values at the Venturi tube inlet, in the throat and downstream of the outlet, respectively; ΔP(x) represents irreversible pressure drop, T₁ is flow temperature of the produced oil-water mixture at the Venturi tube inlet, ρ, cₚ, μ_{JT} and η—are density, heat capacity, Joule-Thompson coefficient and an adiabatic coefficient of the oil-water mixture, respectively.

The temperature of the oil-water mixture downstream of the Venturi tube outlet (where V=V₁) is determined by the total irreversible pressure loss and the Joule-Thompson fluid coefficient:

T₃≈T₁+(−μ_{JT})·(P₁−P₃) (2)

In the case of a homogeneous mixture of oil and water (which is typical of the flow passing through the Venturi tube throat in oil wells), the density of the oil-water mixture, the adiabatic coefficient and the Joule-Thompson coefficient depend on the water cut (γ) (see equations 3-5), and changes in the temperature of the oil-water mixture in the Venturi tube can be used to determine a water fraction in the mixture.

$$\begin{matrix}{ρ}_{m} = γ \cdot {ρ}_{w} + ( 1 - γ ) \cdot {ρ}_{o} & ( 3 ) \\ {η}_{m} = \frac{γ \cdot {ρ}_{w} {c}_{pw} \cdot {η}_{w} + ( 1 - γ ) \cdot {ρ}_{o} {c}_{po} \cdot {η}_{o}}{γ \cdot {ρ}_{w} {c}_{pw} + ( 1 - γ ) \cdot {ρ}_{o} {c}_{po}} & ( 4 ) \\ {μ}_{JTm} = \frac{γ \cdot {ρ}_{w} {c}_{pw} \cdot {μ}_{JTw} + ( 1 - γ ) \cdot {ρ}_{o} {c}_{po} \cdot {μ}_{JTo}}{γ \cdot {ρ}_{w} {c}_{pw} + ( 1 - γ ) \cdot {ρ}_{o} {c}_{po}} & ( 5 )\end{matrix}$$

Since oil is a complex mixture of various hydrocarbons, the adiabatic coefficient and the Joule-Thompson coefficient in each particular case should be determined by the results of laboratory studies of the relationship between pressure, volume and temperature using oil samples from specific wells. FIGS. 2 and 3 show the examples of the relationship of these coefficients versus pressure (at a temperature of 80° C.) for certain hydrocarbons present in the oil. It can be seen from these diagrams that, for example, at a pressure of 150 bar, the Joule-Thompson coefficient of oil is approximately 1.5-2 times greater than for water, and the adiabatic coefficient is 4-6 times greater.

FIG. 4 shows the calculated relationship of changes in the flow temperature downstream of the Venturi tube outlet versus water cut. The calculations were performed for Joule-Thompson coefficient values for water −0.02 K/bar and for oil −0.04 K/bar. The pressure difference between the inlet and the throat of the Venturi tube P₁-P₂ was in the range of 0.7-0.8 bar. The flow velocity at the Venturi tube inlet is 2 m/s. This velocity is typical for borehole control devices in highly productive wells.

It can be seen in this figure that depending on the water content, the change in temperature difference T₁-T₃ is about 7 mK, which is a measure measurable by modern devices located in the borehole and can be used for estimating the water content in the oil-water mixture.

High-sensitive temperature sensors should be installed at the following points: 1-2 diameters of the Venturi tube before the Venturi tube constriction (for measuring the inlet temperature) and 10-20 diameters of the Venturi tube upstream of the Venturi tube throat (to measure the temperature rise caused by the Joule-Thomson effect).

Another, much stronger, heat effect that can be used for determining the water cut, is heating of the Venturi tube wall, caused by viscous dissipation. Numerical calculations demonstrate that, due to the viscous dissipation effect in the oil-water mixture flow, the temperature in the boundary layer near the Venturi tube wall and the wall temperature in the neck can significantly exceed the temperature T₁ at the Venturi tube inlet.

FIG. 5 shows calculated radial distribution of velocities (dashed lines) and temperature at the beginning of the constriction and in the Venturi tube throat. Calculations were performed for the velocity of the oil-water mixture at the Venturi tube inlet of 3.5 m/s. It can be seen in the figure that the thickness of the dynamic boundary layer in this case is about 1 mm. The thickness of the thermal boundary layer is significantly less (less than 0.3 mm), and temperature rise of the wall reaches 650 mK.

The temperature rise of the wall in the Venturi tube throat depends on the oil-water mixture composition and can be used for estimating the water cut. FIG. 6 shows calculated relationship of the temperature rise of the walls versus water cut. The calculations were performed for an average flow velocity at the Venturi tube inlet of 2 m/s and oil viscosity 3 times greater than water viscosity. It can be seen from this figure that the temperature of the walls strongly depends on the water cut: 150 mK for pure water and 580 mK for oil. Due to a much stronger temperature signal, in this case, a more accurate estimate of the water cut of the mixture can be obtained than from the temperature rise due to the Joule-Thomson effect downstream of the Venturi tube outlet.

The temperature of the walls in the Venturi tube throat depends on the geometry of the Venturi tube, the production rate of the well, the oil characteristics and the water content. Based on simulation of the Venturi tube (using the methods of computational fluid dynamics) and laboratory experiments, a set of preliminary calculations for various oil characteristics should be prepared. These preliminary calculations should be used to estimate the water cut in the wells.

Another thermal effect that can be used to determine the phase composition of the produced oil-water mixture is adiabatic heating or adiabatic cooling of the oil-water mixture caused by sudden pressure changes δP in the borehole (for example, in case of production rate change or production shut off):

δT=ηₘ·δP (6)

These changes are determined by the adiabatic coefficient of the mixture (4). FIG. 7 shows the calculated relationship of the amplitude of adiabatic temperature changes versus water cut for the pressure change δP=10 bar.

Specific feature of the proposed method for determining the phase composition of the produced oil-water mixture by its adiabatic heating/cooling consists in using temperature measurements downstream of the Venturi tube outlet, which ensures reliable homogenization of the flow, thereby reducing uncertainty associated with location of a temperature meter in a separate phase, but not in the homogenized mixture.

According to the method, it is proposed to evaluate the water cut of the produced oil-water mixture basing on the high-precision measurement of pressure and temperature of the flow at the Venturi tube inlet (P₁, T₁) and the measurement of wall temperature T_{2w} and pressure P₂ in the Venturi tube throat; the measurements can also be supplemented by the measurement of the flow pressure and flow temperature downstream of the Venturi tube outlet (P₃, T₃) during oil production. The calculation of water cut is performed according to formulas (3)-(6), by taking into account the characteristics of the oil produced.

Calculation of the water cut by heating of walls in the Venturi tube throat is performed according to the values of P₁, T₁, P₂, T_{2w}, by comparing the results of calculations with the corresponding preliminary calculations based on the characteristics of the oil produced.

It is also possible to perform the measurements using all the mentioned meters of temperature variation of the flow through the Venturi tube, caused by sudden changes in pressure in case of the production rate change or production shut off. The calculation of the water content is performed according to formula (7), by taking into account the dependence of the adiabatic coefficient (5) on the water cut and the properties of the oil produced.

The proposed method can provide a reliable estimate of the water cut of the oil-water mixture produced from any selected segment of the well, with the help of a Venturi tube placed in a borehole by obtaining several values pertaining to one and the same water cut. This makes it possible to reduce uncertainty of the final water cut value by using a joint analysis of all or only some of the indicated measurements, by taking into account the corresponding measurement errors and the temperature signal values.

If a segment with a high water cut of the produced oil-water mixture is identified, production from this segment of the well is ceased.

1. A method for determining a water cut of an oil-water mixture produced from an oil well, the method comprising:
placing at least one Venturi tube in a well, through which the oil-water mixture produced from a selected well segment enters a borehole,
measuring pressure at an inlet of the Venturi tube and in a throat of the Venturi tube during production process,
measuring by temperature sensors a flow temperature of the produced oil-water mixture at the Venturi tube inlet and a temperature of a wall of the Venturi tube in the Venturi tube throat, and
based on the results of the pressure and temperature measurements, determining the water cut of the oil-water mixture produced from the selected well segment.

2. The method of claim 1, comprising additionally determining the water cut of the produced oil-water mixture using the measurement results of the pressure and temperature of the produced oil-water mixture downstream of the Venturi tube outlet.

3. The method of claim 1, comprising determining the water cut of the produced oil-water mixture using measurements of temperature variations of the produced fluid in the Venturi tube when the pressure in the well changes due to production rate change or production shut off.

4. The method of claim 1, wherein for measuring the temperature at the Venturi tube inlet the temperature sensors are used arranged at a distance of 1 to 2 diameters of the Venturi tube before the Venturi tube constriction.

5. The method of claim 2, wherein for measuring the temperature downstream of the Venturi tube outlet the temperature sensors are used arranged at a distance of 10-20 diameters of the Venturi tube upstream of the the Venturi tube throat.
